(19) United States
(12) Patent Application Publication
(10) Pub. No.: US 2004/0267110 A1
(43) Pub. Date: Dec. 30, 2004

(54) Method for detection of vulnerable plaque

(76) Inventor: Patrice Tremble, Santa Rosa, CA (US)

Correspondence Address: MEDTRONIC VASCULAR, INC., 3576 Unocal Place, Santa Rosa, CA, 95403, US

(21) Appl. No.: 10/867,357

(22) Filed: Jun. 14, 2004

Related U.S. Application Data
(60) Provisional application No. 60/477,982, filed on Jun. 12, 2003.

(51) Int. Cl. A61B 5/05; A61B 6/00

(52) U.S. Cl. 600/407; 600/476

(57) ABSTRACT

A method of detecting vulnerable plaque is provided. The method includes stopping a heart from beating, preparing a bloodless field, and inspecting the bloodless field for vulnerable plaque using Optical Coherent Tomography.

### RELATED APPLICATIONS

This application claims the benefit of U.S. Provisional Patent Application 60/477,982 filed Jun. 12, 2003.

### FIELD OF THE INVENTION

This invention relates to methods for detecting vulnerable plaque, especially during stopped heart procedures.

### BACKGROUND OF THE INVENTION

Heart disease, specifically coronary artery disease, is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged expanding the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap, some of which are only two micrometers thick or less. The lipid pool is believed to be formed as a result of pathological process involving low density lipoprotein (LDL), macrophages, and the inflammatory process. The macrophages oxidize the LDL producing foam cells. The macrophages, foam cells, and associated endothelial cells release various substances, such as tumor necrosis factor, tissue factor, and matrix proteinases. These substances can result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection (e.g., diagnosis and localization) of vulnerable plaques. One strategy involves the measurement of temperature within a blood vessel. For example, vulnerable plaque tissue temperature is generally elevated compared to healthy vascular tissue. Measurement of this temperature discrepancy may allow detection of the vulnerable plaque.

Another detection strategy involves labeling vulnerable plaque with a marker. The marker substance may be specific for a component and/or characteristic of the vulnerable plaque. For example, the marker may have an affinity for the vulnerable plaque, more so than for healthy tissue. Detection of the marker may thus allow detection of the vulnerable plaque. Alternatively, the marker may not necessarily have an affinity for the vulnerable plaque, but will simply change properties while associated with the vulnerable plaque. The property change may be detected and thus allow detection of the vulnerable plaque.

Direct imaging of the vulnerable plaque would provide a new approach to detection of vulnerable plaque. However, imaging of the vulnerable plaque is difficult due to the opacity of the blood stream. The flow of blood in the vicinity of the vulnerable plaque renders conventional direct imaging technologies difficult.

Accordingly, it would be desirable to provide a method for detecting vulnerable plaque that would overcome the aforementioned and other disadvantages.

### SUMMARY OF THE INVENTION

One aspect of the present invention provides a method of detecting vulnerable plaque. A heartbeat is stopped. After stopping the heart beat, a substantially bloodless field is prepared, and the bloodless field is inspected for vulnerable plaque using an Optical Coherent Tomography (“OCT”) system.

In another aspect of the present invention, a heart is stopped, and a catheter is positioned in a vessel of a body. The catheter comprises an occluding balloon, a saline solution supply and an OCT imaging system. A substantially bloodless field is prepared by inflating the occluding balloon upstream of the bloodless field to occlude the vessel. After occluding the vessel, the bloodless field is flushed with a saline solution. The method continues with an inspection for vulnerable plaque using an OCT system.

The foregoing, and other, features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims in equivalence thereof.

### BRIEF DESCRIPTION OF THE DRAWINGS

FIG. 1 is a flowchart depicting one embodiment of a method in accordance with one aspect of the present invention;

FIG. 2 is a flowchart depicting one embodiment of a method in accordance with one aspect of the present invention; and,

FIG. 3 is a flowchart depicting one embodiment of a method in accordance with one aspect of the present invention.

### DETAILED DESCRIPTION OF THE PRESENTLY PREFERRED EMBODIMENTS

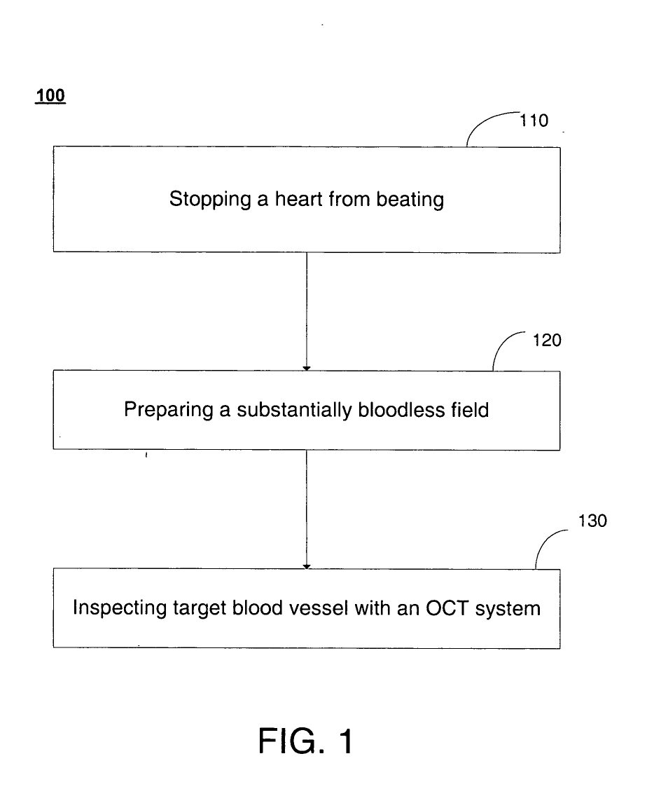

FIG. 1 shows a flowchart of one embodiment of a method for detecting vulnerable plaque in accordance with the present invention at 100.

Method 100 begins at block 110 wherein a heart is stopped. In one example, the heart is stopped in conjunction with another medical procedure, such as cardiac bypass surgery, cardiac valve surgery, a fluoroscopic procedure, a cardiac procedure not involving a coronary bypass and not involving cardiac valve surgery, a vascular procedure, a neurosurgical procedure, an electrophysiology procedure, an ablation procedure, an endovascular procedure, a pulmonary procedure, an aneurysm repair, an imaging procedure, a CAT scan procedure, a MRI procedure, a genetic therapy, a cellular therapy, a cancer therapy, a radiation therapy, a transplantation procedure, a coronary angioplasty procedure, an atherectomy procedure, a procedure that requires precise control of cardiac motion, a procedure that requires precise control of bleeding, a port-access procedure, an endoscopic procedure, a sternotomy procedure, a thoracotomy procedure, a robotic procedure and cardiac surgery requiring that the heart be still. The term “medical procedure” may further mean any one or more medical or surgical procedures such as, for example cardiac surgery, performed with or without cardiopulmonary bypass (CPB) circuits, heart valve repair, heart valve replacement, Maze procedures, revascularization procedures, transmyocardial revascularization (TMR) procedures, percutaneous myocardial revascularization (PMR) procedures, anastomosis procedures, fluoroscopic procedures, beating heart surgery, neurosurgery, brain surgery, electrophysiology procedures, diagnostic and therapeutic procedures, ablation of arrhythmias, endovascular procedures, treatment of the liver, spleen, heart, lungs, and major blood vessels, aneurysm repair, imaging procedures of the heart and great vessels, pharmacological therapies, drug delivery procedures, gene therapies, genetic, cellular, tissue and/or organ manipulation or transplantation procedures, coronary angioplasty procedures, placement or delivery of coated or noncoated stents, and atherosclerotic plaque manipulation and/or removal procedures.

The medical procedure may be non-invasive, minimally invasive and/or invasive. The medical procedure may entail a port-access approach, a partial or total endoscopic approach, a sternotomy approach or a thoracotomy approach. The medical procedure may include the use of various mechanical stabilization devices or techniques as well as various robotic or imaging systems.

In one method, the heart may be temporarily slowed or intermittently stopped for short periods of time to permit the surgeon to accomplish the required surgical task and yet still allow the heart itself to supply oxygenated blood to the body. For example, stimulation of the vagus nerve in order to temporarily and intermittently slow or stop the heart is disclosed in U.S. Pat. No. 6,006,134 entitled “Method and device for electronically controlling the beating of a heart using venous electrical stimulation of nerve fibers”, Dec. 21, 1999, to Hill and Junkman. This patent is assigned to Medtronic, Inc.

At block 110, the heart may be stopped using nerve stimulation. The nerve may be the vagus nerve, a carotid sinus nerve or a fat pad. Techniques for stopping the heart with this method are well known to those of ordinary skill in the art. Method 100 may also include use of a nerve stimulator (not shown). In one embodiment, the nerve stimulator (not shown) may be used to electrically manipulate cardiac rhythm by stimulating the vagus nerve. This vagal stimulation may produce asystole (slowing or stopping of the heart's beating.) Once this induced asystole is stopped, i.e. once the vagal stimulation is stopped, the heart may be allowed to return to its usual cardiac rhythm. Alternatively, the heart may be paced with an electrical pacing system, thereby maintaining a normal cardiac output. Electrical pacing may be selectively and intermittently stopped to allow a surgeon to perform a surgical procedure during asystole.

It is known that stimulation of the vagus nerve can reduce the sinus rate, as well as prolong AV (atrioventricular) conduction time or, if stimulation energies are high enough, induce AV node block. Use of vagal nerve stimulation to treat supraventricular arrhythmias and angina pectoris is disclosed in the article “Vagal Tuning” by Bilgutay et al., Journal of Thoracic and Cardiovascular Surgery, Vol. 56, No. 1, July, 1968, pp. 71-82. It is also known that stimulation of the carotid sinus nerve produces a similar result, as disclosed in the article “Carotid Sinus Nerve Stimulation in the Treatment of Angina Pectoris and Supraventricular Tachycardia” by Braunwald et al., published in California Medicine, Vol. 112, pp. 41-50, March, 1970.

As set forth in “Functional Anatomy of the Cardiac Efferent Innervation” by Randall et al., in Neurocardiology, edited by Kulbertus et al, Futura Publishing Co., 1988, direct surgical excision of the fat pad associated with the SA (sinoatrial) node affects the functioning of the SA node without significantly affecting the AV node. Similarly, excision of the fat pad associated with the AV node affects functioning of the AV node without significantly affecting the SA node.

As set forth in the article “Parasympathetic Postganglionic Pathways to the Sinoatrial Node”, Bluemel et al., Am. J. Physiol. 259, (Heart Circ. Physiol. 28) H1504-H1510, 1990, stimulation of the fat pad associated with the SA node results in slowing of the sinus rate without the accompanying prolongation of AV conduction time which normally results from vagal nerve stimulation. The article also indicates that stimulation of the fat pad associated with the AV node is believed to produce corresponding effects limited to the AV node, i.e., extension of the AV conduction time without concurrent slowing of the sinus rate.

As set forth in the article “Neural Effects on Sinus Rate and Atrial Ventricular Conduction Produced by Electrical Stimulation From a Transvenous Electrode Catheter in the Canine Right Pulmonary Artery” by Cooper et al., published in Circulation Research, Vol. 46, No. 1, January, 1980, pp. 48-57, the fat pads associated with both the AV node and the SA node may be stimulated by means of electrodes located in the right pulmonary artery. The results obtained include both a depression of the sinus rate and a prolongation of the AV conduction time in response to continuous stimulation at 2-80 Hertz at up to 50 milliamps.

Alternatively, at block 110, stopping a heart from beating may comprise administering a drug. The drug may be any appropriate drug that will slow or stop a heart beat. These drugs may also produce reversible asystole of a heart while maintaining the ability of the heart to be electrically paced. Other drugs may be administered for a variety of functions and purposes as described below. Drugs may be delivered at any appropriate time during the medical procedure, for example, at the beginning of the procedure, intermittently during the procedure, continuously during the procedure or following the procedure.

Drugs, drug formulations or compositions suitable for administration to a patient during a medical procedure may include a pharmaceutically acceptable carrier or solution in an appropriate dosage. There are a number of pharmaceutically acceptable carriers that may be used for delivery of various drugs, for example, via direct injection, oral delivery, suppository delivery, transdermal delivery, epicardial delivery and/or inhalation delivery. Pharmaceutically acceptable carriers include a number of solutions, preferably sterile, for example, water, saline, Ringer's solution and/or sugar solutions such as dextrose in water or saline. Other possible carriers that may be used include sodium citrate, citric acid, amino acids, lactate, mannitol, maltose, glycerol, sucrose, ammonium chloride, sodium chloride, potassium chloride, calcium chloride, sodium lactate, and/or sodium bicarbonate. Carrier solutions may or may not be buffered.

Drug formulations or compositions may include antioxidants or preservatives such as ascorbic acid. They may also be in a pharmaceutically acceptable form for parenteral administration, for example to the cardiovascular system, or directly to the heart, such as intracoronary infusion or injection. Drug formulations or compositions may comprise agents that provide a synergistic effect when administered together. A synergistic effect between two or more drugs or agents may reduce the amount that normally is required for therapeutic delivery of an individual drug or agent. Two or more drugs may be administered sequentially or simultaneously. Drugs may be administered via one or more bolus injections and/or infusions or combinations thereof. The injections and/or infusions may be continuous or intermittent. Drugs may be administered, for example, to a coronary artery and/or vein, a pulmonary artery and/or vein, the right atrium and/or ventricle, the left atrium and/or ventricle, the aorta, the AV node, and/or the coronary sinus. Drugs may be administered or delivered via intravenous, intracoronary and/or intraventricular administration in a suitable carrier. Examples of arteries that may be used to deliver drugs to the AV node include the AV node artery, the right coronary artery, the right descending coronary artery, the left coronary artery, the left anterior descending coronary artery and Kugel's artery. Besides being delivered locally, drugs may be delivered systemically, for example, via oral, transdermal, intranasal, suppository or inhalation methods. Drugs also may be delivered via a pill, a spray, a cream, an ointment or a medicament formulation.

Drugs may be delivered via a drug delivery device that may comprise a catheter, such as a drug delivery catheter or a guide catheter, a patch, such as a transepicardial patch that slowly releases drugs directly into the myocardium, a cannula, a pump and/or a hypodermic needle and syringe assembly. A drug delivery catheter may include an expandable member, e.g., a low-pressure balloon, and a shaft having a distal portion, wherein the expandable member is disposed along the distal portion. A catheter for drug delivery may comprise one or more lumens and may be delivered endovascularly via insertion into a blood vessel, e.g., an artery such as a femoral, radial, subclavian or coronary artery. The catheter can be guided into a desired position using various guidance techniques, e.g., flouroscopic guidance and/or a guiding catheter or guide wire techniques.

Drugs may be delivered via an iontophoretic drug delivery device placed on the heart. In general, the delivery of ionized drugs may be enhanced via a small current applied across two electrodes. Positive ions may be introduced into the tissues from the positive pole, or negative ions from the negative pole. The use of iontophoresis may markedly facilitate the transport of certain ionized drug molecules. For example, lidocaine hydrochloride may be applied to the heart via a drug patch comprising the drug. A positive electrode could be placed over the patch and current passed. The negative electrode would contact the heart or other body part at some desired distance point to complete the circuit. One or more of the electrodes may also be used as nerve stimulation electrodes or as cardiac stimulation electrodes.

The two divisions of the autonomic nervous system that regulate the heart have opposite functions. First, the adrenergic or sympathetic nervous system increases heart rate by releasing epinephrine and norepinephrine. Second, the parasympathetic system also known as the cholinergic nervous system or the vagal nervous system decreases heart rate by releasing acetylcholine. Catecholamines such as norepinephrine (also called noradrenaline) and epinephrine (also called adrenaline) are agonists for beta-adrenergic receptors. An agonist is a stimulant biomolecule or agent that binds to a receptor.

Beta-adrenergic receptor blocking agents compete with beta-adrenergic receptor stimulating agents for available beta-receptor sites. When access to beta-receptor sites are blocked by receptor blocking agents, also known as beta-adrenergic blockade, the chronotropic or heart rate, inotropic or contractility, and vasodilator responses to receptor stimulating agents are decreased proportionately. Therefore, beta-adrenergic receptor blocking agents are agents that are capable of blocking beta-adrenergic receptor sites.

Since beta-adrenergic receptors are concerned with contractility and heart rate, stimulation of beta-adrenergic receptors, in general, increases heart rate, the contractility of the heart and the rate of conduction of electrical impulses through the AV node and the conduction system.

Drugs, drug formulations and/or drug compositions that may be used according to this invention may include any naturally occurring or chemically synthesized (synthetic analogues) beta-adrenergic receptor blocking agents. Beta-adrenergic receptor blocking agents or β-adrenergic blocking agents are also known as beta-blockers or β-blockers and as class II antiarrhythmics.

The term “beta-blocker” appearing herein may refer to one or more agents that antagonize the effects of beta-stimulating catecholamines by blocking the catecholamines from binding to the beta-receptors. Examples of beta-blockers include, but are not limited to, acebutolol, alprenolol, atenolol, betantolol, betaxolol, bevantolol, bisoprolol, carterolol, celiprolol, chlorthalidone, esmolol, labetalol, metoprolol, nadolol, penbutolol, pindolol, propranolol, oxprenolol, sotalol, teratolol, timolol and combinations, mixtures and/or salts thereof.

The effects of administered beta-blockers may be reversed by administration of beta-receptor agonists, e.g., dobutamine or isoproterenol.

The parasympathetic or cholinergic system participates in control of heart rate via the SA node, where it reduces heart rate. Other cholinergic effects include inhibition of the AV node and an inhibitory effect on contractile force. The cholinergic system acts through the vagal nerve to release acetylcholine, which, in turn, stimulates cholinergic receptors. Cholinergic receptors are also known as muscarinic receptors. Stimulation of the cholinergic receptors decreases the formation of cAMP. Stimulation of cholinergic receptors generally has an opposite effect on heart rate compared to stimulation of beta-adrenergic receptors. For example, beta-adrenergic stimulation increases heart rate, whereas cholinergic stimulation decreases it. When vagal tone is high and adrenergic tone is low, there is a marked slowing of the heart (sinus bradycardia). Acetylcholine effectively reduces the amplitude, rate of increase and duration of the SA node action potential. During vagal nerve stimulation, the SA node does not arrest. Rather, pacemaker function may shift to cells that fire at a slower rate. In addition, acetylcholine may help open certain potassium channels thereby creating an outward flow of potassium ions and hyperpolarization. Acetylcholine also slows conduction through the AV node.

Drugs, drug formulations and/or drug compositions that may be used according to this invention may include any naturally occurring or chemically synthesized (synthetic analogues) cholinergic agent. The term “cholinergic agent” appearing herein may refer to one or more cholinergic receptor modulators or agonists. Examples of cholinergic agents include, but are not limited to, acetylcholine, carbachol (carbamyl choline chloride), bethanechol, methacholine, arecoline, norarecoline and combinations, mixtures and/or salts thereof.

Drugs, drug formulations and/or drug compositions that may be used according to this invention may include any naturally occurring or chemically synthesized cholinesterase inhibitor. The term “cholinesterase inhibitor” appearing herein may refer to one or more agents that prolong the action of acetylcholine by inhibiting its destruction or hydrolysis by cholinesterase. Cholinesterase inhibitors are also known as acetylcholinesterase inhibitors. Examples of cholinesterase inhibitors include, but are not limited to, edrophonium, neostigmine, neostigmine methylsulfate, pyridostigmine, tacrine and combinations, mixtures and/or salts thereof.

There are ion-selective channels within certain cell membranes. These ion selective channels include calcium channels, sodium channels and/or potassium channels. Therefore, other drugs, drug formulations and/or drug compositions that may be used according to this invention may include any naturally occurring or chemically synthesized calcium channel blocker. Calcium channel blockers inhibit the inward flux of calcium ions across cell membranes of arterial smooth muscle cells and myocardial cells. Therefore, the term “calcium channel blocker” appearing herein may refer to one or more agents that inhibit or block the flow of calcium ions across a cell membrane. The calcium channel is generally concerned with the triggering of the contractile cycle. Calcium channel blockers are also known as calcium ion influx inhibitors, slow channel blockers, calcium ion antagonists, calcium channel antagonist drugs and as class IV antiarrhythmics. A commonly used calcium channel blocker is verapamil.

Administration of a calcium channel blocker, e.g., verapamil, generally prolongs the effective refractory period within the AV node and slows AV conduction in a rate-related manner, since the electrical activity through the AV node depends significantly upon the influx of calcium ions through the slow channel. A calcium channel blocker has the ability to slow a patient's heart rate, as well as produce AV block. Examples of calcium channel blockers include, but are not limited to, amiloride, amlodipine, bepridil, diltiazem, felodipine, isradipine, mibefradil, nicardipine, nifedipine (dihydropyridines), nickel, nimodinpine, nisoldipine, nitric oxide (NO), norverapamil and verapamil and combinations, mixtures and/or salts thereof. Verapamil and diltiazem are very effective at inhibiting the AV node, whereas drugs of the nifedipine family have a lesser inhibitory effect on the AV node. Nitric oxide (NO) indirectly promotes calcium channel closure. NO may be used to inhibit contraction. NO may also be used to inhibit sympathetic outflow, lessen the release of norepinephrine, cause vasodilation, decrease heart rate and decrease contractility. In the SA node, cholinergic stimulation leads to formation of NO.

Other drugs, drug formulations and/or drug compositions that may be used according to this invention may include any naturally occurring or chemically synthesized sodium channel blocker. Sodium channel blockers are also known as sodium channel inhibitors, sodium channel blocking agents, rapid channel blockers or rapid channel inhibitors. Antiarrhythmic agents that inhibit or block the sodium channel are known as class I antiarrhythmics, examples include, but are not limited to, quinidine and quinidine-like agents, lidocaine and lidocaine-like agents, tetrodotoxin, encainide, flecainide and combinations, mixtures and/or salts thereof. Therefore, the term “sodium channel blocker” appearing herein may refer to one or more agents that inhibit or block the flow of sodium ions across a cell membrane or remove the potential difference across a cell membrane. For example, the sodium channel may also be totally inhibited by increasing the extracellular potassium levels to depolarizing hyperkalemic values, which remove the potential difference across the cell membrane. The result is inhibition of cardiac contraction with cardiac arrest (cardioplegia). The opening of the sodium channel (influx of sodium) is for swift conduction of the electrical impulse throughout the heart.

Other drugs, drug formulations and/or drug compositions that may be used according to this invention may include any naturally occurring or chemically synthesized potassium channel agent. The term “potassium channel agent” appearing herein may refer to one or more agents that impact the flow of potassium ions across the cell membrane. There are two major types of potassium channels. The first type of channel is voltage-gated and the second type is ligand-gated. Acetylcholine-activated potassium channels, which are ligand-gated channels, open in response to vagal stimulation and the release of acetylcholine. Opening of the potassium channel causes hyperpolarization, which decreases the rate at which the activation threshold is reached. Adenosine is one example of a potassium channel opener. Adenosine slows conduction through the AV node. Adenosine, a breakdown product of adenosine triphosphate, inhibits the AV node and atria. In atrial tissue, adenosine causes the shortening of the action potential duration and causes hyperpolarization. In the AV node, adenosine has similar effects and may also decrease the action potential amplitude and the rate of increase of the action potential. Adenosine is also a direct vasodilator by its actions on the adenosine receptor on vascular smooth muscle cells. In addition, adenosine acts as a negative neuromodulator, thereby inhibiting release of norepinephrine. Class III antiarrhythmic agents also known as potassium channel inhibitors lengthen the action potential duration and refractoriness by blocking the outward potassium channel to prolong the action potential. Amiodarone and d-sotalol are both examples of class III antiarrhythmic agents.

Potassium is the most common component in cardioplegic solutions. High extracellular potassium levels reduce the membrane resting potential. Opening of the sodium channel, which normally allows rapid sodium influx during the upstroke of the action potential, is therefore inactivated because of a reduction in the membrane resting potential. For example, the combination of drugs and vagal stimulation may be used as a cardioplegic agent in a variety of medical procedures.

Drugs, drug formulations and/or drug compositions that may be used in accordance with this invention may comprise one or more of any naturally occurring or chemically synthesized beta-blocker, cholinergic agent, cholinesterase inhibitor, calcium channel blocker, sodium channel blocker, potassium channel agent, adenosine, adenosine receptor agonist, adenosine deaminase inhibitor, dipyridamole, monoamine oxidase inhibitor, digoxin, digitalis, lignocaine, bradykinin agents, serotoninergic agonist, antiarrythmic agents, cardiac glycosides, local anesthetics and combinations or mixtures thereof. Digitalis and digoxin both inhibit the sodium pump. Digitalis is a natural inotrope derived from plant material, while digoxin is a synthesized inotrope. Dipyridamole inhibits adenosine deaminase, which breaks down adenosine.

Drugs, drug formulations and/or drug compositions capable of reversibly suppressing autonomous electrical conduction at the SA and/or AV node, while still allowing the heart to be electrically paced to maintain cardiac output may be used according to this invention.

In one embodiment, the cardiac asystole produced in accordance with the present invention is reversible, e.g., chemically such as by the administration of atropine or by natural forces. Beta-adrenergic stimulation or administration of calcium solutions may be used to reverse the effects of a calcium channel blocker such as verapamil. Agents that promote heart rate and/or contraction may be used in a preferred embodiment of the present invention. For example, dopamine, a natural catecholamine, is known to increase contractility. Positive inotropes are agents that specifically increase the force of contraction of the heart. Glucagon, a naturally occurring hormone, is known to increase heart rate and contractility. Glucagon may be used to reverse the effects of a beta-blocker since its effects bypass the beta receptor. Forskolin is known to increase heart rate and contractility. As mentioned earlier, epinephrine and norepinephrine naturally increase heart rate and contractility. Thyroid hormone, phosphodiesterase inhibitors and prostacyclin, a prostaglandin, are also known to increase heart rate and contractility. In addition, methylxanthines are known to prevent adenosine from interacting with its cell receptors.

At block 120, a bloodless field is prepared. A bloodless field describes the area that is to be inspected for vulnerable plaque. This area will be in a vessel of a body, and will be predetermined. After the heart was stopped at block 110, new blood supply should not flow through the bloodless field. However, it may be desirable to further prepare the field by removing any blood remaining in the field. In order to practice the invention, the area to be inspected for vulnerable plaque must not have any blood flow. As is known to those of ordinary skill in the art, OCT (optical coherent tomography) imaging is not easily achievable through blood flow.

In one embodiment of a method in accordance with the current invention, the bloodless field of the vessel is flushed with a saline solution. The target area may be flushed using a catheter-based flushing apparatus, or the flushing may be performed pericardially with the use of needles. Those of ordinary skill in the art will readily recognize that other appropriate solutions may be used for flushing the bloodless field. Flushing the bloodless field may improve the quality of the OCT imaging and detection, but in some patients, natural blood flow may be sufficient to remove enough blood to allow accurate imaging and detection of the vulnerable plaque. In other embodiments, any biocompatible fluid that may facilitate the detection of vulnerable plaque while using OCT imaging may be used in the place of a saline solution.

In another embodiment of a method in accordance with the current invention, an occluding balloon is first inflated upstream from the bloodless field to stop the flow of blood. After occluding the vessel, an area downstream from the occlusion may be flushed with a saline solution, or other appropriate solution.

The occluding balloon may be part of a catheter delivery system. The saline solution may also be part of a catheter delivery system, or may be epicardially delivered. Where the saline solution is delivered epicardially, appropriate delivery methods, such as via a needle, may be used. Indeed, any tool that is capable of preventing blood flow downstream may be used in place of an occluding balloon, such as a vascular clamp.

Devices for occluding blood streams are known to those of ordinary skill in the art. In such embodiments, those of ordinary skill in the art will recognize a number of techniques to occlude the blood vessel. In one example, the products of Velocimed Inc., of Minneapolis, Minn. may be used. Velocimed manufactures catheter-based balloon devices that expand upstream from a target site to occlude blood flow. Other products include the Percusurge Guard Wire that is deployed downstream from the target site. The Percusurge Guard Wire is available from Traatek, Inc. of Fort Lauderdale, Fla. Additionally, other tools or instruments may be used, including manual occlusion or the insertion of objects to interrupt blood flow. Such objects may include spongy materials.

After occlusion of the blood vessel, no fresh blood should flow to the area to be imaged. However, some blood may remain in the desired field of view. Although this volume of blood may not interfere with the operation of the OCT apparatus, it may be desirable to flush the blood vessel with a saline solution to present an optimal image. As described in method 100, the vessel may be flushed using a catheter-based system, or a pericardial system.

At block 130, a target blood vessel is imaged with an optical coherence tomography (“OCT”) system to detect the presence of vulnerable plaque. As is well known, an OCT apparatus is an optical imaging apparatus that can perform micron-resolution, cross-sectional imaging (also referred to as tomographic imaging) of biological tissue. As is known in the art, OCT apparatus work by comparing the optical path of two radiation streams to create an optical interference signal. The optical interference signal may be used to create an image of the biological tissue that is to be imaged. Combining data from serial scans forms a cross-sectional image of the tissue. Where the biological tissue to be imaged is obscured by flowing liquid, such as blood, the image quality may be degraded. The OCT system may be delivered to the target site using a catheter system, or may be delivered pericardially. Catheter-based OCT devices are well-known in the art. Where a patient is already undergoing an open procedure, such as a coronary bypass, it may be preferred to deliver the OCT device directly to the target to be imaged. However, where a patient is not undergoing an open procedure, it may be preferred to utilize a catheter-based system. For an example of catheter-based OCT systems, see U.S. Pat. No. 6,546,272 to MacKinnon, issued Apr. 8, 2003.

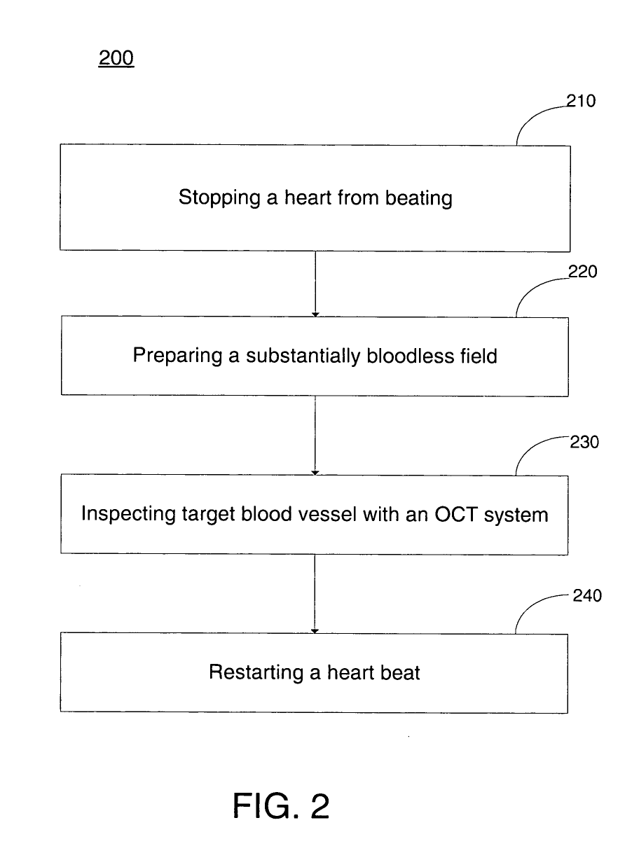

FIG. 2 is a flowchart describing another method in accordance with the current invention. A heart is stopped from beating at block 210. Methods for this step are described above, as in block 110. A substantially bloodless field is prepared at block 220, in accordance with the description above for block 120. The bloodless field is inspected with an OCT system at block 230, in accordance with the description above for block 130.

The heartbeat is restarted at block 240. Techniques for restarting the heart are known to those of ordinary skill in the art. The heart may be restarted by use of nerve stimulation, or by administering a drug. Techniques for restarting the heart by nerve stimulation are similar to those described above. Drugs to restart the heart are also known to those of ordinary skill in the art.

It may be desirable to perform any number of medical procedures after detecting the vulnerable plaque, but before restarting the heart. Furthermore, it also may be desirable to treat the vulnerable plaque. It may be desirable to attempt to detect vulnerable plaque in other vessels or in other locations of the same vessel. Those of ordinary skill in the art will recognize that many medical procedures may be appropriate after detecting vulnerable plaque with the method disclosed herein.

It may be desired to perform this method during the course of another medical procedure. During many medical procedures, the heart is stopped for medical reasons other than a desire to inspect a bloodless field for vulnerable plaque. Such medical procedures may include cardiac bypass surgery, cardiac valve surgery, a fluoroscopic procedure, a cardiac procedure not involving a coronary bypass and not involving cardiac valve surgery, a vascular procedure, a neurosurgical procedure, an electrophysiology procedure, an ablation procedure, an endovascular procedure, a pulmonary procedure, an aneurysm repair, an imaging procedure, a CAT scan procedure, an MRI procedure, a genetic therapy, a cellular therapy, a cancer therapy, a radiation therapy, a transplantation procedure, a coronary angioplasty procedure, a stent delivery procedure, an atherectomy procedure, a procedure that requires precise control of cardiac motion, a procedure that requires precise control of bleeding, a port-access procedure, an endoscopic procedure, a sternotomy procedure, a thoracotomy procedure and a robotic procedure and cardiac surgery requiring that the heart be still. The method of this invention may be readily practiced during such procedures.

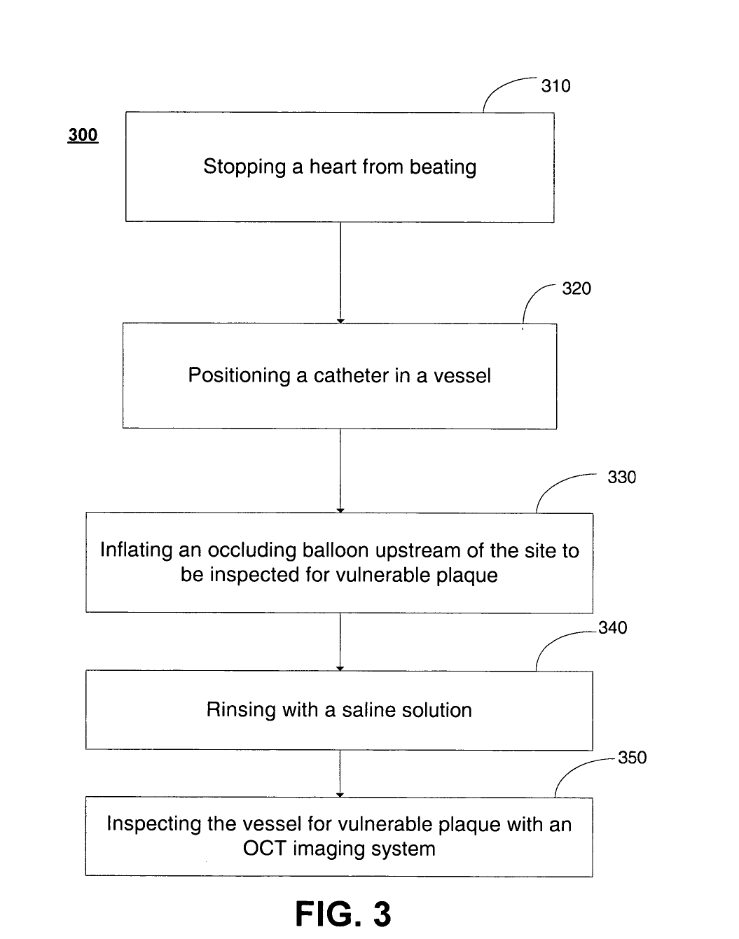

FIG. 3 presents another embodiment of a method in accordance with the instant invention. Method 300 begins at block 310, where a heart is stopped from beating, in accordance with the description for block 110 of method 100. At block 320, a catheter comprising an occluding balloon, saline delivery system and an OCT imaging device is positioned in a vessel. Catheter delivery systems for delivery of an occluding balloon are well known to those of ordinary skill in the art, as are catheter delivery systems for a saline flush. Catheter based OCT systems are described in U.S. Pat. No. 6,546,272 to MacKinnon, issued Apr. 8, 2003. Catheter guidance techniques are also well known to those of ordinary skill in the art. Sample techniques may include the use of a guide wire or use of radio-opaque imaging techniques. Saline solution delivery systems are also well known in the art, and may comprise a reservoir of a saline solution, a pump, a lumen in the catheter to convey the saline solution to the bloodless field and a way to deliver the saline solution to the bloodless field.

Method 300 continues at block 330. At block 330, a bloodless field is prepared. In this embodiment of the invention, the target blood vessel is first occluded upstream from the bloodless field using the occluding balloon. After occlusion of the vessel, the bloodless field is flushed with a saline solution to clear the blood at block 340. After creation of the bloodless field, method 300 continues to block 350, where the bloodless field is inspected for vulnerable plaque with an OCT imaging system. Block 350 is similar to blocks 130 and 230 of methods 100 and 200 respectively.

Any of the methods disclosed herein may comprise use of a catheter, such as a drug delivery catheter, a guide catheter, a catheter OCT system, or a catheter occluding system, or a catheter for flushing a blood vessel with a saline solution. These catheters may include an expandable member, e.g., a low-pressure balloon, and a shaft having a distal portion, wherein the expandable member is disposed along the distal portion. These catheters may comprise one or more lumens and may be delivered endovascularly via insertion into a blood vessel, e.g., an artery such as a femoral, radial, subclavian or coronary artery. The catheter can be guided into a desired position using various guidance techniques, e.g., flouroscopic guidance and/or a guiding catheter or guide wire techniques. In one embodiment, one catheter is used to deliver all necessary components to practice the invention—the OCT imaging device, saline solution, and an occluding device (if used). If a drug is used to stop the heart beat, the drug may be administered with a patch, such as a transepicardial patch that slowly releases drugs directly into the myocardium, a cannula, a pump and/or a hypodermic needle and syringe assembly.

Where a drug is used to stop the heartbeat, the drug may also be administered with an iontophoretic drug delivery device placed on the heart. In general, the delivery of ionized drugs may be enhanced via a small current applied across two electrodes. Positive ions may be introduced into the tissues from the positive pole, or negative ions from the negative pole. The use of iontophoresis may markedly facilitate the transport of certain ionized drug molecules. For example, lidocaine hydrochloride may be applied to the heart via a drug patch comprising the drug. A positive electrode could be placed over the patch and current passed. The negative electrode would contact the heart or other body part at some desired distance point to complete the circuit.

The drug delivery system used to practice this invention may be any suitable system for delivering a drug that will stop the heartbeat.

All, or a portion, of the drug delivery system may be placed in any suitable manner for application of drugs to the heart. In one embodiment, the drugs are administered directly to a vessel of the heart. The drugs may be administered invasively or non-invasively. In one embodiment, all or a portion of the drug delivery system is implanted adjacent the target area of the heart. Alternatively, all or a portion of drug delivery system is removably applied to the target area of the heart. For example, the system may comprise a vasodilative cream manually applied to the target site followed by a vasoconstrictive spray manually applied to the site. Alternatively, the drug administering system may comprise a guidable or steerable mechanism, such as a catheter, which allows its position to be adjusted during the medical procedure. The drug administering device may be positioned endoscopically to suitable location, such as on or near a target coronary artery and/or vein, a pulmonary artery and/or vein, the right atrium and/or ventricle, the left atrium and/or ventricle, the aorta, the AV node, and/or the coronary sinus. The drug delivery system may also be positioned to administer or deliver drugs via intravenous, intracoronary and/or intraventricular administration in a suitable carrier. Examples of arteries that may be used to deliver drugs to the AV node include the AV node artery, the right coronary artery, the right descending coronary artery, the left coronary artery, the left anterior descending coronary artery and Kugel's artery.

All or a portion of the drug delivery system may also be placed in any suitable manner for application of drugs to another area of the body such as the leg or another limb. For example, the system may be placed to apply vasoactive substances to a saphenous vein to be harvested or to any other suitable graft vessel. In one embodiment, system is placed to deliver drugs directly to a suitable vessel to be imaged. The drug delivery system may be placed invasively or non-invasively. In one embodiment, the drug delivery system is implanted adjacent the graft vessel. Alternatively, drug delivery system is removably applied to the graft vessel.

AC Current, DC Current, disposable batteries and re-chargeable batteries may power the drug delivery system. The drug delivery system may comprise a surgeon controlled switch box. A switch, or all of the drug delivery system may also be incorporated in or on one of the surgeon's instruments, such as surgical site retractor, or any other location easily and quickly accessed by the surgeon for delivery of drugs by the surgeon. The switch may be, for example, a hand switch, a foot switch, or a voice-activated switch comprising voice-recognition technologies.

The drug delivery system may be operably connected to a nerve stimulator or a cardiac stimulator. Software controlling drug delivery system may be designed to automatically deliver drugs while a nerve stimulator or a cardiac stimulator is operating.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A method of detecting vulnerable plaque in a vessel of a body, comprising:
stopping a heart within the body from beating;
preparing a substantially bloodless field in a portion of the vessel while the heart is stopped;
inspecting the vessel for vulnerable plaque in the substantially bloodless field in the portion of the vessel, wherein the inspection for vulnerable plaque is performed using an OCT imaging system, while the heart remains stopped.

2. The method of claim 1 wherein preparing a substantially bloodless field comprises removing blood from the vessel.

3. The method of claim 2 wherein removing blood from the blood vessel comprises a using a saline flush.

4. The method of claim 2 wherein removing blood from the blood vessel comprises occluding the vessel upstream from the bloodless field.

5. The method of claim 4 further comprising using a saline flush after occluding the vessel upstream from the bloodless field, and prior to inspecting the vessel for vulnerable plaque.

6. The method of claim 1 wherein stopping a heart within the body from beating comprises a method selected from the group consisting of: vagus nerve stimulation, carotid sinus nerve stimulation, and stimulation of a fat pad.

7 The method of claim 1 wherein stopping a heart within the body from beating comprises administering a drug selected from the group consisting of: a beta-blocker, a cholinergic agent, a cholinesterase inhibitor, a calcium channel blocker, a sodium channel blocker, a potassium channel agent, adenosine, an adenosine receptor agonist, an adenosine deaminase inhibitor, dipyridamole, a monoamine oxidase inhibitor, digoxin, digitalis, lignocaine, a bradykinin agent, a serotoninergic agonist, an antiarrythmic agent, a cardiac glycoside, a local anesthetic, atropine, a calcium solution, an agent that promotes heart rate, an agent that promotes heart contractions, dopamine,
a catecholamine, an inotrope glucagon, a hormone, forskolin, epinephrine, norepinephrine, thyroid hormone, a phosphodiesterase inhibitor, prostacyclin, prostaglandin and a methylxanthine.

8. The method of claim 1 wherein the OCT imaging system comprises a catheter delivery system.

9. The method of claim 1 wherein the OCT imaging system comprises a pericardial system.

10. The method of claim 1 wherein stopping the heart from beating occurs during the course of a medical procedure.

11. The method of claim 10 wherein the heart is stopped during a medical procedure chosen from the group consisting of cardiac bypass surgery, cardiac valve surgery, a fluoroscopic procedure, a cardiac procedure not involving a coronary bypass and not involving cardiac valve surgery, a vascular procedure, a neurosurgical procedure, an electrophysiology procedure, an ablation procedure, an endovascular procedure, a pulmonary procedure, an aneurysm repair, an imaging procedure, a CAT scan procedure, a MRI procedure, a genetic therapy, a cellular therapy, a cancer therapy, a radiation therapy, a transplantation procedure, a coronary angioplasty procedure, a stent delivery procedure, an atherectomy procedure, a procedure that requires precise control of cardiac motion, a procedure that requires precise control of bleeding, a port-access procedure, an endoscopic procedure, a sternotomy procedure, a thoracotomy procedure, a robotic procedure, and cardiac surgery requiring that the heart be still.

12. The method of claim 1, further comprising starting a heart beat after inspecting the vessel for vulnerable plaque.

13. A method of detecting vulnerable plaque in a vessel of a body, comprising:
stopping a heart within the body from beating;
positioning a catheter in the vessel, the catheter comprising an occluding balloon, a saline solution delivery system, and an OCT imaging system;
preparing a substantially bloodless field in a portion of the vessel while the heart is stopped by first inflating the occluding balloon upstream from the bloodless field and then flushing the bloodless field with the saline solution; and
inspecting the vessel for vulnerable plaque in the substantially bloodless field in the portion of the vessel, wherein the inspection for vulnerable plaque is performed using an OCT imaging system, while the heart remains stopped.

14. The method of claim 13 further comprising starting the heart after inspecting the vessel for vulnerable plaque.

15. The method of claim 13 wherein stopping the heart from beating occurs during the course of a medical procedure.

16. The method of claim 15 wherein the medical procedure is chosen from the group consisting of cardiac bypass surgery, cardiac valve surgery, a fluoroscopic procedure, a cardiac procedure not involving a coronary bypass and not involving cardiac valve surgery, a vascular procedure, a neurosurgical procedure, an electrophysiology procedure, an ablation procedure, an endovascular procedure, a pulmonary procedure, an aneurysm repair, an imaging procedure, a CAT scan procedure, a MRI procedure, a genetic therapy, a cellular therapy, a cancer therapy, a radiation therapy, a transplantation procedure, a coronary angioplasty procedure, a stent delivery procedure, an atherectomy procedure, a procedure that requires precise control of cardiac motion, a procedure that requires precise control of bleeding, a port-access procedure, an endoscopic procedure, a sternotomy procedure, a thoracotomy procedure, a robotic procedure, and cardiac surgery requiring that the heart be still.

17. The method of claim 13 wherein stopping a heart within the body from beating comprises a method selected from the group consisting of: vagus nerve stimulation, carotid sinus nerve stimulation, and stimulation of a fat pad.

18. The method of claim 13 wherein stopping a heart within the body from beating comprises administering a drug selected from the group consisting of: a beta-blocker, a cholinergic agent, a cholinesterase inhibitor, a calcium channel blocker, a sodium channel blocker, a potassium channel agent, adenosine, an adenosine receptor agonist, an adenosine deaminase inhibitor, dipyridamole, a monoamine oxidase inhibitor, digoxin, digitalis, lignocaine, a bradykinin agent, a serotoninergic agonist, an antiarrythmic agent, a cardiac glycoside, a local anesthetic, atropine, a calcium solution, an agent that promotes heart rate, an agent that promotes heart contractions, dopamine, a catecholamine, an inotrope glucagon, a hormone, forskolin, epinephrine, norepinephrine, thyroid hormone, a phosphodiesterase inhibitor, prostacyclin, prostaglandin and a methylxanthine.
